(19) United States
(12) Patent Application Publication
(10) Pub. No.: US 2011/0208074 A1
(43) Pub. Date: Aug. 25, 2011

(54) Digital EKG machine & portable wireless printer

(76) Inventor: Pamela Ann Anderson, Springfield, IL (US)

(21) Appl. No.: 12/660,327

(22) Filed: Feb. 25, 2010

(51) Int. Cl. A61B 5/0402

(52) U.S. Cl. 600/509

(57) ABSTRACT

The digital EKG machine is used to provide an accurate reading of the voltage between pairs of these electrodes, and the muscle activity that they measure from different directions. The main components of the digital EKG machine are the info red laser lens, which is the reading source for the EKG machine. It will track each parts of the body as it glad slowly to different area of accurate testing.

The extended lens is used to make sure each section is properly recording on the miniature CD. The main components of the portable printer are the CD slot, which will hold the miniature CD. The alpha keyboard is used to record the patient name and date of birth or accuracy. The display monitor will allow the doctors and nurses to ensure accurate results.

### BACKGROUND OF THE INVENTION

I created the Digital EKG Sensor, and Portable Wireless Laptop Printer because I knew there had to be an easier way of getting an EKG reading from a patient, besides using all of the leads and sticky electrodes plugs. This invention will allow the medical community to produce an accurate and quicker EKG reading. The Portable Wireless Laptop Printer will not only print and display the EKG reading but it can be used to email the EKG reading to other medical facilities since it will have internet compatibilities.

### SUMMARY OF THE INVENTION

This invention will cater to the medical community, and the electronic world. The Digital EKG Sensor and Portable Wireless Laptop Printer will pave the way traditional EKG reading is performed. Not only will this invention make it easier to prepare a patient for an accurate EKG reading, it will allow more room in the examination area and provide comfort during preparation time for the patient. The Digital EKG Sensor and Portable Wireless Laptop Printer will provide internal and external view of the scanned body areas. The doctors and nurses will be able to see which parts of the body were scanned and which areas were not, from the display screen on the Portable Wireless Laptop Printer and the extended lens of the EKG Sensor.

this Digital EKG Sensor sole purpose is to produce a digital EKG reading of the entire body without all the wired leads, and sticky electrode, which will cut preparation time in half. The ideal of the invention is for the Digital EKG Sensor and Portable Wireless Laptop Printer to work together without touching each other. Each one will have a sensor chip that will accurately read each scanned body area and ensure nothing will be missed. Both machines are lightweight and portable. The medical/electronic community will be able to travel at a moment notices. Portability is the way business is conducted in today's world.

this invention will be described in reference to the application Ser. No. 29/348,960 filed on May 3, 2010 entitled “Anderson Portable EKG Machine”. Within this description I will describe the enter workings of the devices that were presented in application Ser. No. 29/348,960. Each figure of the drawers provides detail information relating to the hardware and how each piece will function while in use. The Digital EKG Sensor and Portable Laptop Printer will serve the medical community; because it's lightweight energy efficient, portable, and compact in a carrying case.

with this invention the medical community will save time, and space due to the compact size. No more hassle with cable leads that sometime get tangled during preparation. The Ambulance staff can use the Digital EKG Sensor and Portable Wireless Laptop Printer with ease. Not only will they be able to get an accurate EKG reading, they will be able to email the reading to the hospital in route a head of time so the medical staff will know what situation they will be facing once the patient arrives at the medical facility.

### BRIEF DESCRIPTION OF THE DRAWINGS

FIG. 1. Shows the side view of the Portable Wireless Laptop Printer. I incorporated this angle to show how deep the storage drawer will be. There will be various components that can be storage in the drawer. Things like: the printer head, motor, belt, stabilizer, bar, feed, stepper motor, power supply, control circuitry, wireless USB port, protected heat thermal, EKG paper, Toner cartridge, EKG paper feed and the wireless electrode with mini cam built in each one. The bottom drawer will also be able to hold pencils, pens, prescription pad and sticky note pad.

FIG. 2. Shows the Digital EKG Sensor, it is oval shape for easy gripping. The Sensor can be used like a computer mouse for scanning the body if needed. The Sensor can detect blood clogs throughout the body. The rotating extendable removable lens is used for viewing purposes. The bottom of the sensor is the most important part of the machine, it the part that is utilize to detect problem areas of the body.

FIG. 3. Shows the full front view of the Portable Laptop Printer. The Portable Laptop will act the same way as any other laptops, with one noticeable different, it will actually print out the EKG reading on paper. The 12.4 inch display 1280×800 WXGA, Glossy bright monitor is be big enough for viewing and small enough to fit on the table where the medical staff can take notes. The utility tray is used for extra storage, things like paper clips, pencils, and pens. It's compact and portable and just right for a medical vehicle.

FIG. 4. Shows the inside of the drawer that is below the laptop printer. The drawer is deep enough to hold all the necessary storage items. The front handle is used to pull the drawer in and out. The laptop itself will remain stabilized while the drawer is being pulled out for use.

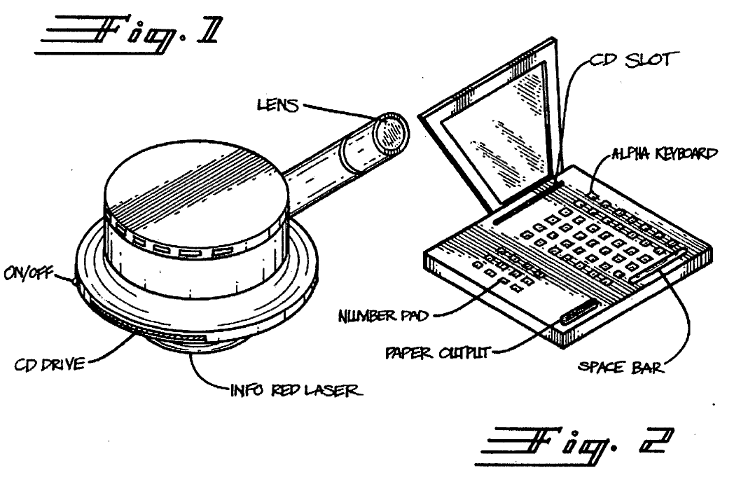

1. The digital EKG Machine and Portable Printer is an easier way to get an EKG reading of the patient body. The preparation time for a digital EKG reading will be cut in half. There will be no need for lead wires and sticky tape that is placed on different areas of the body, and sometime left behind after a procedure. Both piece of equipment are designed to be lightweight and portable for easy storage. Doctors and nurses will love the portability of the digital EKG machine and printer.
Both piece of equipment are environmentally friendly, there are no hazardous materials for disposal. There were no hazardous materials used in the design and manufacture stages. The machines will travel where ever there is a need for an accurate EKG reading. Both machines will revolutionize the way traditional EKG reading is being performed. I Pamela Anderson, claim I am the sole inventor of the Digital EKG machine and portable printer.
